(19) United States
(12) Patent Application Publication
(10) Pub. No.: US 2008/0125384 A1
(43) Pub. Date: May 29, 2008

(54) SIMULTANEOUS SILENCING AND RESTORATION OF GENE FUNCTION

(76) Inventor: Shuewi Yang, Germantown, MD (US)

Correspondence Address: Baker Donelson Bearman Caldwell & Berkowitz, PC;Attn: Kening Li, 6th Floor, 555 11th Street, NW, Washington, DC, 20004, US

(21) Appl. No.: 11/562,281

(22) Filed: Nov. 21, 2006

Related U.S. Application Data
(60) Provisional application No. 60/738,048, filed on Nov. 21, 2005.

(51) Int. Cl. A61K 31/70; C12N 15/87; C12N 15/82; A61P 43/00; C12N 15/00; C07H 21/04

(52) U.S. Cl. 514/44; 435/455; 435/466; 536/24.1; 435/320.1

(57) ABSTRACT

Method and composition for simultaneously silencing an endogenously transcribed polynucleotide sequence in a cell using RNAi and replacing the function of the endogenous sequence with the function of an exogenous polynucleotide sequence. Also provided are methods of treatment based on the above method, and pharmaceutical composition comprising the vectors of the present invention. Further provided are methods for verifying that silencing or knock-down of a gene in a cell is due to siRNA interference. The method and composition of the present invention takes advantage of the sequence specificity of the RNAi gene silencing or knock down mechanism.

### BACKGROUND OF THE INVENTION

The small interfering RNA (siRNA), or RNA inference (RNAi), technique is one of the most significant advances in molecular genetics (see e.g. Fire et al., 1998, Nature 391:806-811; Devroe and Silver, 2004, Expert Opin. Biol. Ther. 4: 319-327; Shi, Trends in Genetics, 2003, 19:9-12; Leung and Whittaker, 2005, Pharmacology & Therapeutics, 107:222-239; and Wadhwa et al, 2004, Current Opinion in Molecular Therapeutics 6:367-372, incorporated herein by reference in their entirety). Its ability to silence the function of a target gene in a sequence-specific manner is widely believed to have the potential of revolutionizing gene function research and treatment methods for many diseases.

Gene silencing and knockdown using RNA interference has become routine. FIG. 1 briefly illustrates the mechanism of the RNAi technology, and FIG. 2 exemplifies a vector-based (e.g. plasmid or viral vector) technique for implementing the technology. In FIG. 2A, the formation of a double-stranded short hairpin RNA (shRNA) is illustrated. The shRNA is subsequently processed to siRNA by the cellular machinery. Short hairpin RNA constructs (shRNA) have advantages over siRNA because the effects of these constructs can lead to a more stable and long-term result.

However, there remains an un-met need, both in research and in clinical applications, for simultaneously eliminating the function of a defective gene in a cell, and restoring the function of the wild type allele of the gene, or replacing the defective gene with the function or another gene.

### SUMMARY OF THE INVENTION

The present invention provides methods and compositions that satisfy the above need. In one embodiment, the present invention provides a method for silencing an endogenously transcribed polynucleotide sequence in a cell and replacing the function of the endogenous sequence with the function of an exogenous polynucleotide sequence, the method comprising: a) introducing into the cell simultaneously i) a first nucleotide sequence coding for siRNA or shRNA against the endogenous polynucleotide under the control of a first promoter; and ii) a second nucleotide sequence coding for exogenous polynucleotide sequence under the control of a second promoter; b) culturing the cell under conditions that the first and second nuclide sequences are transcribed in the cell, wherein the endogenous polynucleotide is a mutated form of the exogenous polynucleotide, and wherein the endogenously transcribed polynucleotide sequence is silenced; and wherein the exogenous polynucleotide is transcribed or expressed.

Preferably, in the above method, the exogenous polynucleotide is a wild-type form of the endogenous polynucleotide, and the function of endogenous polynucleotide is silenced and the wild-type phenotype is restored. In another embodiment, the exogenous sequence encodes the wild type function of the endogenous sequence but further comprises one or more silent mutations such that the exogenous sequence is more divergent from the endogenous sequence at the nucleotide level than the wild type sequence. This sequence divergence allows the knock-down effect of the siRNA encoded by the first nucleotide on the function of the exogenous sequence to be reduced or completely eliminated.

The method of the present invention for silencing an endogenously transcribed polynucleotide sequence in a cell and replacing its function an exogenous polynucleotide sequence may also be accomplished by a) providing a vector which comprises i) a first nucleotide sequence coding for siRNA or shRNA against the endogenous polynucleotide under the control of a first promoter; and ii) a second polynucleotide sequence coding for exogenous polynucleotide sequence under the control of a second promoter; b) introducing the vector into the cell; and c) culturing the cell under conditions that the first and second nucleotide sequences are transcribed in the cell, wherein the endogenously transcribed polynucleotide sequence is silenced; and wherein the exogenous polynucleotide is expressed. In a preferred embodiment, the exogenous polynucleotide is a wild-type form of the endogenous polynucleotide, and wherein the endogenous polynucleotide is silenced and the wild-type phenotype is restored. Alternatively, the exogenous sequence encodes the wild type function of the endogenous sequence and further comprises one or more silent mutations such that the exogenous sequence is more divergent from the endogenous sequence at the nucleotide level than the wild type sequence, such that the knock-down effect of the siRNA encoded by the first nucleotide on the function of the exogenous sequence is reduced.

In preferred embodiments, the endogenous polynucleotide comprises a mutation that causes a deleterious effect to the cell, and the exogenous polynucleotide is capable of eliminating the deleterious effect. For example, the mutation may be a cancer-causing or other disease-causing mutation.

Preferably, the promoter that controls the expression of the restoring sequence is a constitutional promoter, or similar in expression regulation to the endogenous gene, such that the normal function of the endogenous gene is restored, for example in accordance with its gene expression pattern during normal growth or development of the cell or the organism.

The present invention further provides an isolated polynucleotide sequence comprising i) a first nucleotide sequence under the control of a first promoter, wherein the first nucleotide codes for siRNA or shRNA against a coding sequence of a cell; and ii) a second nucleotide sequence under the control of a second promoter, wherein the second nucleotide sequence encodes a variant of the coding sequence, wherein the coding sequence is silenced or its function knocked down and the variant coding sequence is expressed when the isolated polynucleotide sequence is introduced into the cell. Preferably, the coding sequence is a mutant or a defective allele of a wild type sequence and the variant sequence is the wild-type form of the coding sequence. Particularly preferably, the variant sequence may encode the wild type function of the coding sequence yet comprises one or more silent mutations so that the variant sequence is more divergent from the coding sequence at the nucleotide level in comparison to the wild type sequence, such that the knock-down effect of the siRNA encoded by the first nucleotide on the function of the variant sequence is reduced.

The above polynucleotide sequence is preferably a vector, more preferably an expression vector comprising suitable regulatory elements a selection marker and therefor such as a suitable promoter for the expression of the polynucleotide sequence. The vectors may be a HSV, AAV, AV or Lentiviral based vector.

The present invention further provides a method for restoring the function of a mutated gene in a mammal in need thereof, the method comprising administering to the mammal a vector of the present invention as described above, wherein the first nucleotide sequence is under the control of a first promoter and codes for siRNA or shRNA against the mutated gene; and the second nucleotide sequence encodes a functional variant of the mutated gene. Preferably, method according to claim 16, wherein the first and the second promoter are different from each other and each is independent selected from the group consisting of H1, U6, CMV core, full length CMV, EF1 alpha (core), SV40, and beta-actin promoters, or a derivative thereof. The method of the present invention may preferably used to eliminate the effect of a mutated gene that causes a disease or a undesired condition in an organism, such as a mammal especially a human patient.

In another embodiment, the present invention provides a method for verifying that silencing or knock-down of a gene in a cell is due to siRNA interference. In one scenario, the gene comprises at least one 3′- or one 5′-untranslated region (UTR), and the method of the present invention comprises 1) introducing to the cell siRNA or shRNA against the gene based on one or both of the UTRs; 2) determining that the expression of the gene is silenced or knocked down; 3) introducing to the cell a vector that comprises a coding sequence of the gene without the UTR on which the siRNA or shRNA is based, wherein the coding sequence is under the control of a suitable regulatory element, and 4) culturing the cell under conditions to allow the expression of the coding sequence, wherein restoration of the function of the gene is indication that the gene knock-down or silencing is due to the siRNA or shRNA. Alternatively, the siRNA is based on and against the cellular or endogenous sequence, and the method comprises 1) introducing to the cell siRNA or shRNA against the cellular gene; 2) determining that the expression of the cellular gene is silenced or knocked down; 3) introducing to the cell a vector that comprises a polynucleotide sequence coding for the product the gene under the control of a suitable regulatory element, and wherein the polynucleotide sequence comprises silent mutations in comparison to the cellular gene sequence, and is not affected by the siRNA or shRNA, and 4) culturing the cell under conditions to allow the expression of the polynucleotide sequence, wherein restoration of the function of the gene is indication that the gene knock-down or silencing is due to the siRNA or shRNA.

### BRIEF DESCRIPTION OF THE DRAWINGS

FIG. 1 illustrates the mechanism of the RNAi technology.

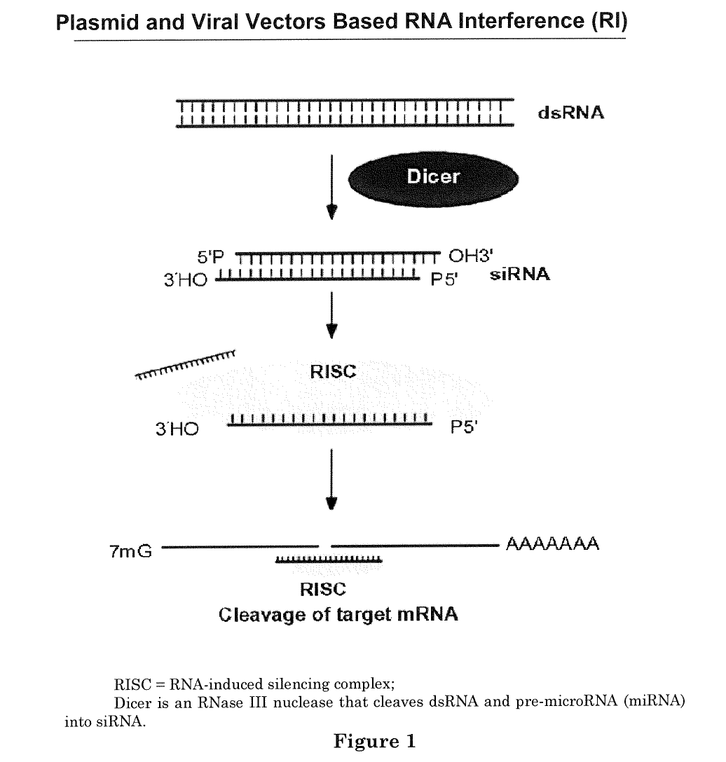

FIG. 2 exemplifies a vector-based technique for implementing the RNAi technology.

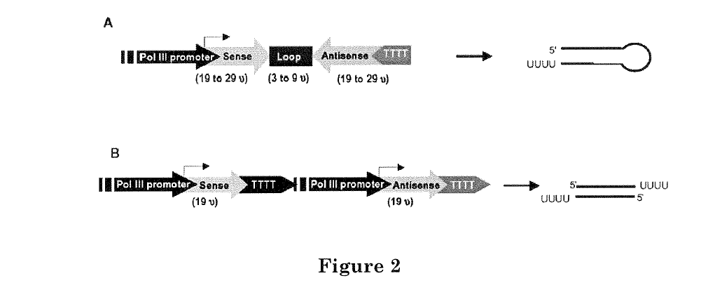

FIG. 3 describes one embodiment of the invention where both the siRNA and the restoring insert are contained in one vector. A vector contains a DNA insert A of mutated region of ORF of the mutated gene X and a DNA insert B of ORF of wild type gene X. DNA insert A encodes siRHA or shRNA and DNA insert B encodes the wild-type protein. The transcription of DNA insert A to siRNA or shRNA will silence the mutated gene X that causes the disease. The transcription and translation of DNA insert B to a wild-type protein shall restore the normal biological and physiological function of Gene X.

FIG. 4 shows an approach to minimizing the effect of the siRNA on the restoring sequence. A vector contains a DNA insert A of wild type region of the mutated gene X ORF and a DNA insert B with desired silence mutation of wild type gene X. DNA insert A encodes siRNA or shRNA and DNA insert B encodes the wild-type protein of gene X. The transcription of DNA insert A to siRNA or shRNA will silence the mutated gene X that cause the disease. The transcription and translation of a wild-type ORF to a wild-type protein for normal biological and physiological function.

FIG. 5 depicts a method that provides confirmation or validation that the siRNA or shRNA has silenced the target gene, wherein the silencing siRNA or shRNA is based on the 5′- or 3′-un-translated region (UTR) of the transcribed sequence.

FIG. 6 depicts a method that provides confirmation or validation that the siRNA or shRNA has silenced the target gene, wherein the silencing siRNA or shRNA is based on a form of the endogenous, to-be-silenced gene, while the restoring sequence differs from the endogenous at the nucleotide level but is functionally identical or equivalent (i.e. a silent mutant).

### DESCRIPTION OF THE INVENTION

The present invention provides a novel method, and compositions based thereupon and used therefor, for simultaneously (1) knock-out, silencing or knock-down the function of an endogenous transcribed sequence or coding sequence or an endogenous gene, and (2) replacing it or restoring the function of, a wild-type gene or an exogenous coding sequence. The method of the present invention may be used for therapeutic purposes for treating a disease that is caused by one or more mutations in a gene.

It is well-known that many human and animal diseases are caused by gene mutations. For example, many cancers such as breast cancer, neurobromatosis, acute myeloid leukemia (AML), and chronic myeloid leukemia (CML), are linked to genetic mutations. Other diseases, such as but not limited to cystic fibrosis, neurofibromatosis, sickle cell anemia are also known to be caused by genetic mutations.

The method of the present invention is also suitable for validation of siRNA silencing of a target gene by demonstrating that the silenced gene function can be successfully restored.

In a preferred embodiment, the endogenous sequence contains one or more mutations that cause aberration in its function which aberration may be the cause of a diseased state of an organism, such as a human patient. The method of the present invention introduces to the cells of the patient simultaneously a sequence that, via the siRNA mechanism, would silence the endogenous sequence, as well as a coding sequence that does not contain the mutation, which coding sequence is under the proper control of a suitable regulatory element (e.g. a promoter) and will restore, or replace, the function of the endogenous sequence to, or with, the non-mutated or wild-type state, thereby providing therapy and treatment of the disease state caused by the mutation.

Two embodiments of the above method are described below. In one embodiment, a bi-molecular (or inter-molecule) approach is employed, wherein siRNA or shRNA based on the region of the open reading frame (ORF) which contains the mutation(s) is obtained, e.g. by chemical synthesis, and is introduced to the cell that contains the mutation simultaneously with a vector that has been engineered to express the wild type ORF which does not contain the mutation(s). Alternatively, the siRNA may also be encoded by a vector engineered to encode the siRNA when the vector is introduced into the cell. The siRNA will silence the function of the mutated ORF, but will have no effect on the wild type ORF. This way, whatever adverse effect the mutated ORF may have on the cell is eliminated, and its role that may be necessary for cell survival or proper function is simultaneously restored by the vector/construct, under the control of a suitable promoter or other regulatory elements appropriate for its function and regulation.

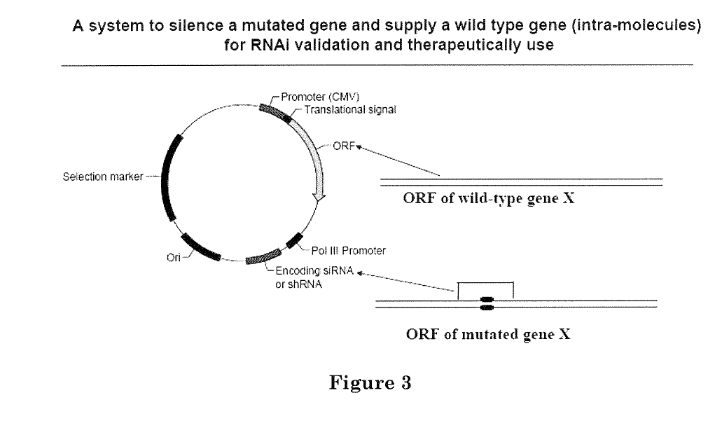

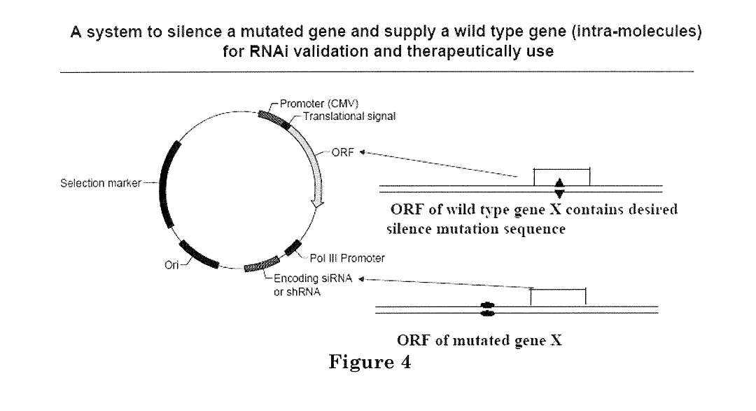

Another embodiment of the present invention, a single-molecule (or intra-molecule) approach, is illustrated in FIGS. 3 and 4. In FIG. 3, both the siRNA and the restoring insert, e.g. a wild-type ORF, are engineered to be contained in one vector, which is introduced to the cell which contains the mutation. Transcription of the siRNA and the wild-type ORF in the cell will achieve the simultaneous silencing or knock-down of the mutated gene, and the restoration of the wild-type function.

FIG. 4 shows an approach to minimizing the effect of the siRNA on the restoring sequence. Specifically, the restoring sequence is engineered to contain silent mutations that do not affect its function or level of expression, but imparts a further sequence divergence from to the mutated gene or the to-be-silenced endogenous gene. Because siRNA silences its target based on sequence identity, the more the sequence divergence between the siRNA molecules and the target sequence, the less the silencing effect. Accordingly, the siRNA will have minimal inhibitive effect on the restoring gene's expression or function.

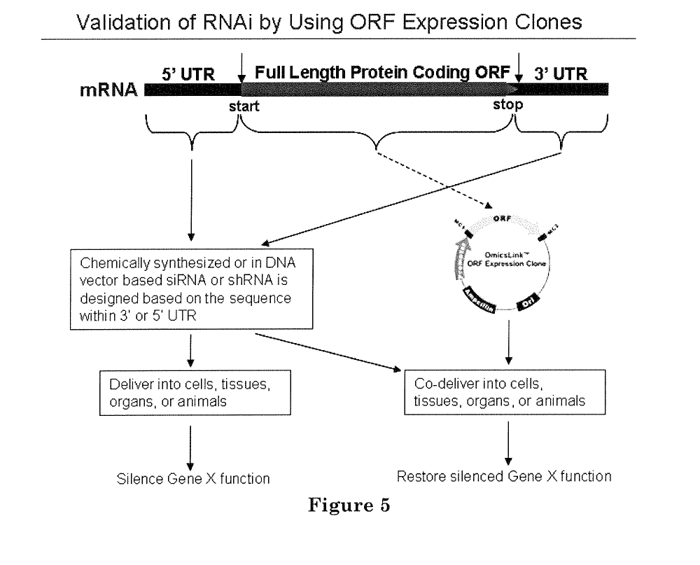

According to another embodiment of the present invention, a method is provided that provides confirmation or validation that the siRNA or shRNA indeed has silenced the target gene. FIG. 5 explains one embodiment, wherein the siRNA or shRNA is based on the 5′- or 3′-un-translated region (UTR) of the transcribed sequence. These UTR based interfering sequences will cause degradation of the entire transcript and silence the target gene. Depending on the genes that have been silenced, various methods are known in the art and available for verification of the absence of target gene function, for example, by determining the silence of the protein encoded by the gene or otherwise measuring the phenotype of the gene.

In order to verify that such silencing is indeed caused by the siRNA, a vector that encodes the ORF region (without the UTRs) of the transcribed sequence is engineered, and when co-introduced, e.g. by co-transfection, into the target cell, should restore the function of the target gene. Because the siRNA is based on the UTRs, it will not interfere with the expression or function of the ORF contained in the vector.

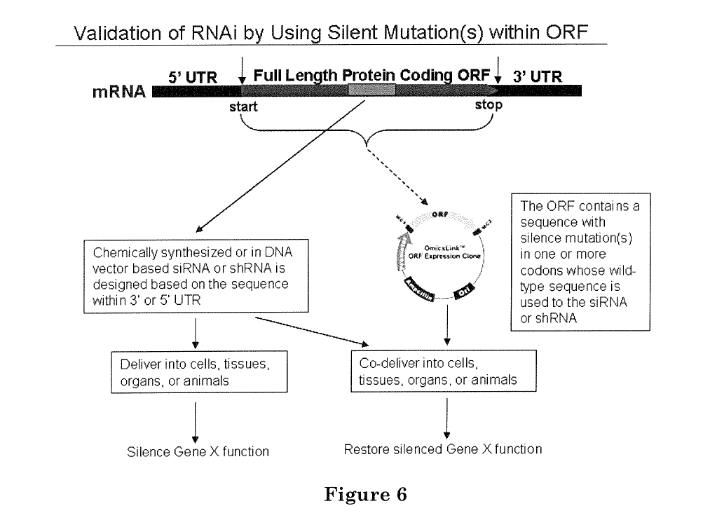

According to a further embodiment of the present invention, the siRNA or shRNA may be based on the ORF region of the target gene. The vector that performs gene function restoration, however, will contain an ORF that comprises, in the region on which the siRNA or shRNA is based, one or more silent mutations (i.e. nucleotide substitutions that do not alter the amino acid encoded by the codon, due to degeneracy of the Genetic Code). An ordinarily skilled artisan will readily recognize that the siRNA will silence the target gene, but when co-introduced into the cell with the vector that contains the silent mutation(s), will not affect the expression of the ORF contained in the vector, allowing the function of the target gene. This is illustrated schematically in FIG. 6.

Recombinant DNA technology, including in vitro recombinant DNA techniques, synthetic techniques, and in vivo genetic recombination, gene therapy techniques. are well- known to those skilled in the art. Such techniques are described in e.g. Sambrook, J. et al. (2001) Molecular Cloning, A Laboratory Manual, Cold Spring Harbor Press, Plainview, N.Y. and Ausubel, F. M. et al. (3^{rd} Ed.); Current Protocols in Molecular Biology, (2006) John Wiley & Sons, New York, N.Y., incorporated herein by reference in their entirety. For example, many expression vectors, i.e., a vector which contains the necessary elements for the transcription and translation of the inserted coding sequence are described in detail. Regulatory elements include for example a promoter, an initiation codon, a stop codon, a mRNA stability regulatory element, and a polyadenylation signal. Expression of a polynucleotide can be assured by (i) constitutive promoters such as the Cytomegalovirus (CMV) promoter/enhancer region, (ii) tissue specific promoters such as the insulin promoter (see, Soria et al., 2000, Diabetes 49:157), SOX2 gene promoter (see Li et al., (1998) Curr. Biol. 8:971-974), Msi-1 promotor (see Sakakibara et al., (1997) J. Neuroscience 17:8300-8312), alpha-cardia myosin heavy chain promoter or human atrial natriuretic factor promotor (Klug et al., (1996) J. clin. Invest 98:216-224; Wu et al., (1989) J. Biol. Chem. 264:6472-6479) or (iii) inducible promoters such as the tetracycline inducible system. Expression vectors can also contain a selection agent or marker gene that confers antibiotic resistance such as the ampecillin, neomycin, hygromycin or puromycin resistance genes.

A preferred promoter for the present invention to control the expression of the siRNA may be the H1 promoter or the U6 promoter, and their derivatives thereof. A promoter for controlling the expression of the restoring sequence may be a promoter selected from the group consisting of the CMV Core promoter, the CMV full length promoter, the EF1 alpha promoter (core), the SV40 promoter, the beta-actin promoter, and their derivatives. The sequence of these promoters are listed in Table 1 below.

TABLE 1

| | |
|---|---|
| H1 | gaacgctgacgtcatcaacccgctccaaggaatcgcgggcccag |
| | tgtcactaggcgggaacacccagcgcgcgtgcgccctggcagga |
| | agatggctgtgagggacaggggagtggcgccctgcaatatttgc |
| | atgtcgctatgtgttctgggaaatcaccataaacgtgaaatgtc |
| | tttggatttgggaatcttataagttctgtatgagaccac |
| | (SEQ ID NO:1) |
| U6 | gggcaggaagagggcctatttcccatgattccttcatatttgca |
| | tatacgatacaaggctgttagagagataattagaattaatttga |
| | ctgtaaacacaaagatattagtacaaaatacgtgacgtagaaag |
| | taataatttcttgggtagtttgcagttttaaaattatgttttaa |
| | aatggactatcatatgcttaccgtaacttgaaagtatttcgatt |
| | tcttggctttatatatcttgtgg |
| | (SEQ ID NO:2) |
| CMV | Tagtaatcaattacggggtcattagttcatagcccatatatgga |
| core | gttccgcgttacataacttacggtaaatggcccgcctggctgac |
| | cgcccaacgacccccgcccattgacgtcaataatgacgtatgtt |
| | cccatagtaacgccaatagggactttccattgacgtcaatgggt |
| | ggagtatttacggtaaactgcccacttggcagtacatcaagtgt |
| | atcatatgccaagtacgccccctattgacgtcaatgacggtaaa |
| | tggcccgcctggcattatgcccagtacatgaccttatgggactt |
| | tcctacttggcagtacatctacgtattagtcatcgctattacca |
| | tggtgatgcggttttggcagtacatcaatgggcgtggatagcgg |
| | tttgactcacggggatttccaagtctccaccccattgacgtcaa |
| | tgggagtttgttttggcaccaaaatcaacgggactttccaaaat |
| | gtcgtaacaactccgccccattgacgcaaatgggcggtaggcgt |
| | gtacggtgggaggtctatataagcagagctggtttagtgaaccg |
| | tcag |
| | (SEQ ID NO:3) |
| Full | ctgcagtgaataataaaatgtgtgtttgtccgaaatacgcgttt |
| length | gagatttctgtcccgactaaattcatgtcgcgcgatagtggtgt |
| CMV | ttatcgccgatagagatggcgatattggaaaaatcgatatttga |
| | aaatatggcatattgaaaatgtcgccgatgtgagtttctgtgta |
| | actgatatcgccatttttccaaaagttgatttttgggcatacgc |
| | gatatctggcgatacgcttatatcgtttacgggggatggcgata |
| | gacgcctttggtgacttgggcgattctgtgtgtcgcaaatatcg |
| | cagtttcgatataggtgacagacgatatgaggctatatcgccga |
| | tagaggcgacatcaagctggcacatggccaatgcatatcgatct |
| | atacattgaatcaatattggccattagccatattattcattggt |
| | tatatagcataaatcaatattggctattggccattgcatacgtt |
| | gtatccatatcataatatgtacatttatattggctcatgtccaa |
| | cattaccgccatgttgacattgattattgactagttattaatag |
| | taatcaattacggggtcattagttcatagcccatatatggagtt |
| | ccgcgttacataacttacggtaaatggcccgcctggctgaccgc |
| | ccaacgacccccgcccattgacgtcaataatgacgtatgttccc |
| | atagtaacgccaatagggactttccattgacgtcaatgggtgga |
| | gtatttacggtaaactgcccacttggcagtacatcaagtgtatc |
| | atatgccaagtacgccccctattgacgtcaatgacggtaaatgg |
| | cccgcctggcattatgcccagtacatgaccttatgggactttcc |
| | tacttggcagtacatctacgtattagtcatcgctattaccatgg |
| | tgatgcggttttggcagtacatcaatgggcgtggatagcggttt |
| | gactcacggggatttccaagtctccaccccattgacgtcaatgg |
| | gagtttgttttggcaccaaaatcaacgggactttccaaaatgtc |
| | gtaacaactccgccccattgacgcaaatgggcggtaggcgtgta |
| | cggtgggaggtctatataagcagagctcgtttagtgaaccgtca |
| | gatcgcctggagacgccatccacgctgttttgacctccatagaa |
| | gacaccgggaccgatccagcctccgcggccgggaacggtgcatt |
| | ggaacgcggattccccgtgccaagagtgacgtaagtaccgccta |
| | tagagtctataggcccacccccttggcttcttatgcatgctata |
| | ctgtttttggcttggggtctatacacccccgcttcctcatgtta |
| | taggtgatggtatagcttagcctataggtgtgggttattgacca |
| | ttattgaccactcccctattggtgacgatactttccattactaa |
| | tccataacatggctctttgcacaactctctttattggctatatg |
| | ccaatacactgtccttcagagactgacacggactctgtattttt |
| | acaggatggggtctcatttattatttacaaattcacatatacaa |
| | caccaccgtccccagtgcccgcagtttttattaaacataacgtg |
| | ggatctccagcgaatctcgggtacgtgttccggacatggggctc |
| | ttctccggtagcggcggagcttctacatccagccctgctcccat |
| | cctcccactcatggtcctcggcagctccttgctcctaacagtgg |
| | aggccagacttaggcacagcacgatgcccaccaccaccagtgtg |
| | cccacaaggccgtggcggtagggtatgtgtctgaaaatgagctc |
| | (SEQ ID NO:4) |
| EF1 | Cctgcagggcccactagtggagccgagagtaattcatacaaaag |
| alpha | gactcgcccctgccttggggaatcccagggaccgtcgttaaact |
| (core) | cccactaacgtagaacccagagatcgctgcgttcccgccccctc |
| | acccgcccgctctcgtcatcactgaggtggagaagagcatgcgt |
| | gaggctccggttcccgtcagtgggcagagcgcacatcgcccaca |
| | gtccccgacaagttggggggaggggtcggcaattgaaccggtgc |
| | ctagagaaggtggcgcggggtaaactgggaaagtgatgtcgtgt |
| | actggctccgcctttttcccgagggtgggggagaaccgtatata |
| | agtgcagtagtcgccgtgaacgttctttttcgcaacgggtttgc |
| | cgtcagaacgcagctgaagcttcgagg |
| | (SEQ ID NO:5) |
| SV40 | Gcgcagcaccatggcctgaaataacctctgaaagaggaacttgg |
| | ttaggtaccttctgaggcggaaagaaccagctgtggaatgtgtg |
| | tcagttagggtgtggaaagtccccaggctccccagcaggcagaa |
| | gtatgcaaagcatgcatctcaattagtcagcaaccaggtgtgga |
| | aagtccccaggctccccagcaggcagaagtatgcaaagcatgca |
| | tctcaattagtcagcaaccatagtcccgcccctaactccgccca |
| | tcccgcccctaactccgcccagttccgcccattctccgccccat |
| | ggctgactaattttttttatttatgcagaggccgaggccgcctc |
| | ggcctctgagctattccagaagtagtgaggaggcttttttggag |
| | gcctaggcttttgcaaaaagctt |
| | (SEQ ID NO:6) |
| beta- | ccaccacctgggtacacacagtctgtgattcccggagcagaacg |
| actin | gaccctgcccacccggtcttgtgtgctactcagtggacagaccc |
| | aaggcaagaaagggtgacaaggacagggtcttcccaggctggct |
| | ttgagttcctagcaccgccccgcccccaatcctctgtggcacat |
| | ggagtcttggtccccagagtcccccagcggcctccagatggtct |
| | gggagggcagttcagctgtggctgcgcatagcagacatacaacg |
| | gacggtgggcccagacccaggctgtgtagacccagcccccccgc |
| | cccgcagtgcctaggtcacccactaacgccccaggccttgtctt |
| | ggctgggcgtgactgttaccctcaaaagcaggcagctccagggt |
| | aaaaggtgccctgccctgtagagcccaccttccttcccagggct |
| | gcggctgggtaggtttgtagccttcatcacgggccacctccagc |
| | cactggaccgctggcccctgccctgtcctggggagtgtggtcct |
| | gcgacttctaagtggccgcaagccacctgactcccccaacacca |
| | cactctacctctcaagcccaggtctctccctagtgacccaccca |
| | gcacatttagctagctgagccccacagccagaggtcctcaggcc |
| | ctgctttcagggcagttgctctgaagtcggcaagggggagtgac |
| | tgcctggccactccatgccctccaagagctccttctgcaggagc |
| | gtacagaacccagggccctggcacccgtgcagaccctggcccac |
| | cccacctgggcgctcagtgcccaagagatgtccacacctaggat |
| | gtcccgcggtgggtggggggcccgagagacgggcaggccggggg |
| | caggcctggccatgcggggccgaaccgggcactgcccagcgtgg |
| | ggcgcgggggccacggcgcgcgcccccagcccccgggcccagca |
| | ccccaaggcggccaacgccaaaactctccctcctcctcttcctc |
| | aatctcgctctcgctctttttttttttcgcaaaaggaggggaga |
| | gggggtaaaaaaatgctgcactgtgcggcgaagccggtgagtga |
| | gcggcgcggggccaatcagcgtgcgccgttccgaaagttgcctt |
| | ttatggctcgagcggccgcggcggcgccctataaaacccagcgg |
| | cgcgacgcgccaccaccgccgagaccgcgtccgccccgcgagca |
| | cagagcctcgcctttgccgatccgccgcccgtccacacccgccg |
| | ccaggtaagcccggccagccgaccg |
| | (SEQ ID NO:7) |

In vivo, ex vivo, or in vitro therapeutic methods are also well-established and well-known to those skilled in the art. For example, many viral vectors are used to deliver exogenous genetic materials to animals, including human patients. The most widely used viral vectors are the retroviruses and adenoviruses, which are used for experimental as well as gene therapy purposes (Kuroki et al., 1999). The high efficiency of adenovirus infection in non replicating cells, the high titer of virus and the high expression of the transduced protein makes this system highly advantageous. Many other vectors are suitable including adenoviral vectors (AV), herpes simplex virus (HSV) type I and type II, and Lentiviral Vectors (e.g. murine leukemia virus (MLV)-based retroviral vector). U.S. Pat. Publication No. 20060019281 provides an exemplary listing of suitable vectors.

As indicated above, siRNA technology is well-known to those skilled in the art. It relates to a process of sequence-specific post-transcriptional gene repression which can occur in eukaryotic cells. In general, this process involves degradation of an mRNA of a particular sequence induced by double-stranded RNA (dsRNA) that is homologous to that sequence. For example, the expression of a long dsRNA corresponding to the sequence of a particular single-stranded mRNA (ss mRNA) will labilize that message, thereby “interfering” with expression of the corresponding gene. Accordingly, any selected gene may be repressed by introducing a dsRNA which corresponds to all or a substantial part of the mRNA for that gene. It appears that when a long dsRNA is expressed, it is initially processed by a ribonuclease III into shorter dsRNA oligonucleotides of as few as 21 to 22 base pairs in length. Accordingly, siRNA may be effected by introduction or expression of relatively short homologous dsRNAs. Indeed the use of relatively short homologous dsRNAs may have certain advantages as discussed below.

Mammalian cells have at least two pathways that are affected by double-stranded RNA (dsRNA). In the siRNA (sequence-specific) pathway, the initiating dsRNA is first broken into short interfering (si) RNAs, as described above. The siRNAs have sense and antisense strands of about 21 nucleotides that form approximately 19 nucleotide siRNAs with overhangs of two nucleotides at each 3′ end. Short interfering RNAs are thought to provide the sequence information that allows a specific messenger RNA to be targeted for degradation. In contrast, the nonspecific pathway is triggered by dsRNA of any sequence, as long as it is at least about 30 base pairs in length.

The nonspecific effects occur because dsRNA activates two enzymes: PKR, which in its active form phosphorylates the translation initiation factor eIF2 to shut down all protein synthesis, and 2′, 5′ oligoadenylate synthetase (2′, 5′-AS), which synthesizes a molecule that activates RNase L, a nonspecific enzyme that targets all mRNAs. The nonspecific pathway may represent a host response to stress or viral infection, and, in general, the effects of the nonspecific pathway are preferably minimized. Significantly, longer dsRNAs appear to be required to induce the nonspecific pathway and, accordingly, dsRNAs shorter than about 30 bases pairs are preferred to effect gene repression by RNAi (see Hunter et al., 1975, J. Biol. Chem. 250:409-17; Manche et al., 1992, Mol. Cell. Biol. 12:5239-48; Minks et al., 1979, J. Biol. Chem. 254:10180-3; and Elbashir et al., 2001, Nature 411:494-8). siRNA has proven to be an effective means of decreasing gene expression in a variety of cell types including HeLa cells, NIH/3T3 cells, COS cells, 293 cells and BHK-21 cells, and typically decreases expression of a gene to lower levels than that achieved using antisense techniques and, indeed, frequently eliminates expression entirely (see Bass, 2001, Nature 411:428-9). In mammalian cells, siRNAs are effective at concentrations that are several orders of magnitude below the concentrations typically used in antisense experiments (Elbashir et al., 2001, Nature 411:494-8).

The double stranded oligonucleotides used to effect RNAi are preferably less than 30 base pairs in length and, more preferably, comprise about 25, 24, 23, 22, 21, 20, 19, 18 or 17 base pairs of ribonucleic acid. Optionally the dsRNA oligonucleotides may include 3′ overhang ends. Exemplary 2-nucleotide 3′ overhangs may be composed of ribonucleotide residues of any type and may even be composed of 2′-deoxythymidine resides, which lowers the cost of RNA synthesis and may enhance nuclease resistance of siRNAs in the cell culture medium and within transfected cells (see Elbashi et al., 2001, Nature 411:494-8).

Longer dsRNAs of 50, 75, 100 or even 500 base pairs or more may also be utilized in certain embodiments of the invention. Exemplary concentrations of dsRNAs for effecting RNAi are about 0.05 nM, 0.1 nM, 0.5 nM, 1.0 nM, 1.5 nM, 25 nM or 100 nM, although other concentrations may be utilized depending upon the nature of the cells treated, the gene target and other factors readily discernable to the skilled artisan.

Exemplary dsRNAs may be synthesized chemically or produced in vitro or in vivo using appropriate expression vectors. Exemplary synthetic RNAs include 21 nucleotide RNAs chemically synthesized using methods known in the art. Synthetic oligonucleotides are preferably deprotected and gel-purified using methods known in the art (see e.g. Elbashir et al., 2001, Genes Dev. 15:188-200). Longer RNAs may be transcribed from promoters, such as T7 RNA polymerase promoters, known in the art. A single RNA target, placed in both possible orientations downstream of an in vitro promoter, will transcribe both strands of the target to create a dsRNA oligonucleotide of the desired target sequence. Any of the above RNA species will be designed to include a portion of nucleic acid sequence represented in a target nucleic acid.

The specific sequence utilized in design of the oligonucleotides may be any contiguous sequence of nucleotides contained within the expressed gene message of the target. Programs and algorithms, known in the art, may be used to select appropriate target sequences. In addition, optimal sequences may be selected utilizing programs designed to predict the secondary structure of a specified single stranded nucleic acid sequence and allowing selection of those sequences likely to occur in exposed single stranded regions of a folded mRNA. Methods and compositions for designing appropriate oligonucleotides may be found, for example, in U.S. Pat. No. 6,251,588, the contents of which are incorporated herein by reference.

Although mRNAs are generally thought of as linear molecules containing the information for directing protein synthesis within the sequence of ribonucleotides, most mRNAs have been shown to contain a number of secondary and tertiary structures. Secondary structural elements in RNA are formed largely by Watson-Crick type interactions between different regions of the same RNA molecule. Important secondary structural elements include intramolecular double stranded regions, hairpin loops, bulges in duplex RNA and internal loops. Tertiary structural elements are formed when secondary structural elements come in contact with each other or with single stranded regions to produce a more complex three dimensional structure. A number of researchers have measured the binding energies of a large number of RNA duplex structures and have derived a set of rules which can be used to predict the secondary structure of RNA (see e.g. Jaeger et al., 1989, Proc. Natl. Acad. Sci. USA 86:7706; and Turner et al.., 1988, Annu. Rev. Biophys. Biophys. Chem. 17:167). The rules are useful in identification of RNA structural elements and, in particular, for identifying single stranded RNA regions which may represent preferred segments of the mRNA to target for siRNA, ribozyme or antisense technologies. Accordingly, preferred segments of the mRNA target can be identified for design of the siRNA mediating dsRNA oligonucleotides as well as for design of appropriate ribozyme and hammerheadribozyme compositions of the invention (see below).

The dsRNA oligonucleotides may be introduced into the cell by transfection with a heterologous target gene using carrier compositions such as liposomes, which are known in the art—e.g. Lipofectamine 2000 (Life Technologies) as described by the manufacturer for adherent cell lines. Transfection of dsRNA oligonucleotides for targeting endogenous genes may be carried out using Oligofectamine (Life Technologies). Transfection efficiency may be checked using fluorescence microscopy for mammalian cell lines after co-transfection of hGFP-encoding pAD3 (Kehlenback et al., 1998, J. Cell Biol. 141:863-74). The effectiveness of the siRNA may be assessed by any of a number of assays following introduction of the dsRNAs. These include Western blot analysis using antibodies which recognize the target gene product following sufficient time for turnover of the endogenous pool after new protein synthesis is repressed, reverse transcriptase polymerase chain reaction and Northern blot analysis to determine the level of existing target mRNA.

Further compositions, methods and applications of siRNA technology are provided in U.S. patent application Ser. Nos. 6,278,039, 5,723,750 and 5,244,805, which are incorporated herein by reference.

We Claim:

1. A method for silencing an endogenously transcribed polynucleotide sequence in a cell and replacing the function of the endogenous sequence with the function of an exogenous polynucleotide sequence, the method comprising:
a) introducing into the cell simultaneously
i) a first nucleotide sequence coding for siRNA or shRNA against the endogenous polynucleotide under the control of a first promoter; and
ii) a second nucleotide sequence coding for exogenous polynucleotide sequence under the control of a second promoter;
b) culturing the cell under conditions that the first and second nuclide sequences are transcribed in the cell,
wherein the endogenous polynucleotide is a mutated form of the exogenous polynucleotide, and
wherein the endogenously transcribed polynucleotide sequence is silenced; and wherein the exogenous polynucleotide is transcribed or expressed.

2. The method according to claim 1, wherein the exogenous polynucleotide is a wild-type form of the endogenous polynucleotide, and wherein the function of endogenous polynucleotide is silenced and the wild-type phenotype is restored.

3. The method according to claim 1, wherein the exogenous sequence encodes the wild type function of the endogenous sequence and comprises one or more silent mutations such that the exogenous sequence is more divergent from the endogenous sequence at the nucleotide level than the wild type sequence, such that the knock-down effect of the siRNA encoded by the first nucleotide on the function of the exogenous sequence is reduced.

4. A method for silencing an endogenously transcribed polynucleotide sequence in a cell and replacing the function of the endogenous sequence with the function of an exogenous polynucleotide sequence, the method comprising:
a) providing a vector which comprises
i) a first nucleotide sequence coding for siRNA or shRNA against the endogenous polynucleotide under the control of a first promoter; and
ii) a second polynucleotide sequence coding for exogenous polynucleotide sequence under the control of a second promoter;
b) introducing the vector into the cell; and
c) culturing the cell under conditions that the first and second nucleotide sequences are transcribed in the cell,
wherein the endogenously transcribed polynucleotide sequence is silenced; and wherein the exogenous polynucleotide is expressed.

5. The method according to claim 4, wherein the exogenous polynucleotide is a wild-type form of the endogenous polynucleotide, and wherein the endogenous polynucleotide is silenced and the wild-type phenotype is restored.

6. The method according to claim 4, wherein the exogenous sequence encodes the wild type function of the endogenous sequence and comprises one or more silent mutations such that the exogenous sequence is more divergent from the endogenous sequence at the nucleotide level than the wild type sequence, such that the knock-down effect of the siRNA encoded by the first nucleotide on the function of the exogenous sequence is reduced.

7. The method according to claim 4, wherein the endogenous polynucleotide comprises a mutation that causes a deleterious effect to the cell, and the exogenous polynucleotide is capable of eliminating the deleterious effect.

8. The method according to claim 7, wherein the mutation is a cancer-causing or other disease causing mutation.

9. The method according to claim 4, wherein the second promoter is a constitutional promoter.

10. An isolated polynucleotide sequence comprising:
i) a first nucleotide sequence under the control of a first promoter, wherein the first nucleotide codes for siRNA or shRNA against a coding sequence of a cell; and
ii) a second nucleotide sequence under the control of a second promoter, wherein the second nucleotide sequence encodes a variant of the coding sequence,
wherein the coding sequence is silenced or its function knocked down and the variant coding sequence is expressed when the isolated polynucleotide sequence is introduced into the cell.

11. The isolated polynucleotide according to claim 10, wherein the coding sequence is a mutant of a wild type sequence, and the variant sequence is the wild-type form of the coding sequence.

12. The isolated polynucleotide according to claim 10, wherein the variant sequence encodes the wild type function of the coding sequence and comprises one or more silent mutations so that the variant sequence is more divergent from the coding sequence at the nucleotide level than a wild type sequence, such that the knock-down effect of the siRNA encoded by the first nucleotide on the function of the variant sequence is reduced.

13. A vector comprising the isolated polynucleotide according to claim 10.

14. The vector according to claim 13, further comprising a selection marker.

15. The vector according to claim 13, which is a HSV, AAV, AV or Lentiviral vector.

16. A method for restoring the function of a mutated gene in a mammal in need thereof, the method comprising administering to the mammal a vector according to claim 13, wherein the first nucleotide sequence is under the control of a first promoter and codes for siRNA or shRNA against the mutated gene; and the second nucleotide sequence encodes a functional variant of the mutated gene.

17. The method according to claim 16, wherein the first and the second promoter are different from each other and each is independent selected from the group consisting of H1, U6, CMV core, Full length CMV, EF1 alpha (core), SV40, and beta-actin promoters, or a derivative thereof.

18. The method according to claim 16, wherein the mutated gene causes a disease or an undesired condition in the mammal.

19. The method according to claim 18, wherein the mammal is a human.

20. A method for verifying that silencing or knock-down of a gene in a cell is due to siRNA interference, wherein the gene comprises at least one 3′- or one 5′-untranslated region (UTR), the method comprising


SEQUENCE LISTING (7 sequences)

SEQ ID NO: 1 - DNA, 215 bp, Artificial Sequence
  H1 promoter
gaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccac

SEQ ID NO: 2 - DNA, 243 bp, Artificial Sequence
  U6 promoter
gggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtgg

SEQ ID NO: 3 - DNA, 576 bp, Artificial Sequence
  CMV core promoter
tagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcag

SEQ ID NO: 4 - DNA, 1848 bp, Artificial Sequence
  full-length CMV promoter
ctgcagtgaataataaaatgtgtgtttgtccgaaatacgcgtttgagatttctgtcccgactaaattcatgtcgcgcgatagtggtgtttatcgccgatagagatggcgatattggaaaaatcgatatttgaaaatatggcatattgaaaatgtcgccgatgtgagtttctgtgtaactgatatcgccatttttccaaaagttgatttttgggcatacgcgatatctggcgatacgcttatatcgtttacgggggatggcgatagacgcctttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgcacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccagcgaatctcgggtacgtgttccggacatggggctcttctccggtagcggcggagcttctacatccagccctgctcccatcctcccactcatggtcctcggcagctccttgctcctaacagtggaggccagacttaggcacagcacgatgcccaccaccaccagtgtgcccacaaggccgtggcggtagggtatgtgtctgaaaatgagctc

SEQ ID NO: 5 - DNA, 215 bp, Artificial Sequence; identical to SEQ ID NO: 1
  EF1 alpha core promoter

SEQ ID NO: 6 - DNA, 419 bp, Artificial Sequence
  SV40 promoter
gcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctt

SEQ ID NO: 7 - DNA, 1301 bp, Artificial Sequence
  Beta-actin promoter
ccaccacctgggtacacacagtctgtgattcccggagcagaacggaccctgcccacccggtcttgtgtgctactcagtggacagacccaaggcaagaaagggtgacaaggacagggtcttcccaggctggctttgagttcctagcaccgccccgcccccaatcctctgtggcacatggagtcttggtccccagagtcccccagcggcctccagatggtctgggagggcagttcagctgtggctgcgcatagcagacatacaacggacggtgggcccagacccaggctgtgtagacccagcccccccgccccgcagtgcctaggtcacccactaacgccccaggccttgtcttggctgggcgtgactgttaccctcaaaagcaggcagctccagggtaaaaggtgccctgccctgtagagcccaccttccttcccagggctgcggctgggtaggtttgtagccttcatcacgggccacctccagccactggaccgctggcccctgccctgtcctggggagtgtggtcctgcgacttctaagtggccgcaagccacctgactcccccaacaccacactctacctctcaagcccaggtctctccctagtgacccacccagcacatttagctagctgagccccacagccagaggtcctcaggccctgctttcagggcagttgctctgaagtcggcaagggggagtgactgcctggccactccatgccctccaagagctccttctgcaggagcgtacagaacccagggccctggcacccgtgcagaccctggcccaccccacctgggcgctcagtgcccaagagatgtccacacctaggatgtcccgcggtgggtggggggcccgagagacgggcaggccgggggcaggcctggccatgcggggccgaaccgggcactgcccagcgtggggcgcgggggccacggcgcgcgcccccagcccccgggcccagcaccccaaggcggccaacgccaaaactctccctcctcctcttcctcaatctcgctctcgctctttttttttttcgcaaaaggaggggagagggggtaaaaaaatgctgcactgtgcggcgaagccggtgagtgagcggcgcggggccaatcagcgtgcgccgttccgaaagttgccttttatggctcgagcggccgcggcggcgccctataaaacccagcggcgcgacgcgccaccaccgccgagaccgcgtccgccccgcgagcacagagcctcgcctttgccgatccgccgcccgtccacacccgccgccaggtaagcccggccagccgaccg